(19) United States
(12) Patent Application Publication
(10) Pub. No.: US 2025/0228768 A1
(43) Pub. Date: Jul. 17, 2025

(54) Intestinal-targeted sustained-release of polyphenols and preparation method and application thereof

(71) Applicant: Zhejiang Gongshang University, Hangzhou (CN)

(72) Inventors: Linglin FU, Hangzhou (CN); Qiaozhi Zhang, Hangzhou (CN); Shiyu Fan, Hangzhou (CN); Junfeng Yin, Hangzhou (CN); Zhengshun Wen, Hangzhou (CN); Yongguan Xu, Hangzhou (CN)

(73) Assignee: Zhejiang Gongshang University, Hangzhou (CN)

(21) Appl. No.: 19/096,628

(22) Filed: Mar. 31, 2025

(30) Foreign Application Priority Data
Dec. 31, 2024 (CN) CN202411974881.7

(51) Int. Cl. A61K 9/00; A61K 36/21; A61K 47/36; A61P 3/04

(52) U.S. Cl. CPC A61K 9/0053; A61K 36/21; A61K 47/36; A61P 3/04

(57) ABSTRACT

The present invention belongs to the field of drug delivery and food and pharmaceutical technology, and specifically relates to resistant starch for intestinal-targeted sustained-release of polyphenols, and a preparation method and application thereof, comprising the following steps: preparing quinoa polyphenol extract; annealing and recrystallizing heat-dispersed high-amylose maize starch in an ethanol solution to obtain single-helix resistant maize starch; self-assembling single-helix resistant maize starch and quinoa polyphenol extract in an alcohol-water system to obtain intestinal-targeted sustained-release of polyphenol/resistant starch. The intestinal-targeted sustained-release of polyphenol/resistant starch prepared by the method of the present invention has a specific starch helical structure that can realize intestinal-targeted controlled release of polyphenols in the content, reduce the loss of phenolic compounds during gastrointestinal digestion, realize the precise and continuous release of polyphenol compounds in the intestinal digestion stage, and prolong the retention time of polyphenols in the intestine, thereby improving the bioavailability of polyphenols.

### TECHNICAL FIELD

The invention belongs to the technical field of drug delivery and food or pharmaceutical composition, and specifically relates to an intestinal-targeted sustained-release polyphenol/resistant starch and a preparation method and application thereof.

### BACKGROUND ART

Polyphenols, as secondary metabolites in plants, are a general term for a large class of heterogeneous phytochemicals and an important component of the human diet. They exhibit outstanding biological activities, such as anti-inflammatory, anti-obesity, and postprandial blood glucose regulation. However, after ingestion, polyphenols undergo extensive chemical degradation and modification in the gastrointestinal tract, which reduces the final concentration of intact polyphenols accumulated in the small intestine and colon. This results in low bioavailability, limiting their ability to fully exert their health benefits. Therefore, it is crucial to improve the stability of polyphenols in the digestive tract to promote their biological properties in the digestive tract. Quinoa, a pseudo-cereal rich in polyphenols, is more beneficial to human health than other plant foods due to its balanced nutritional composition. Studies have shown that quinoa polyphenols have outstanding effects on lowering blood glucose levels and anti-diabetes, especially the activity of pigmented quinoa.

The prior art shows that the use of food-grade encapsulation carriers can improve the biological efficacy of polyphenols. Food-grade biopolymers, such as maize starch, can serve as an external matrix for encapsulating polyphenols. Maize starch, composed of amylose and amylopectin molecules, is one of the most abundant natural polysaccharides. It has the characteristics of safety, low cost, good biocompatibility and biodegradability. However, natural maize starch has high digestibility and is rapidly hydrolyzed by digestive enzymes. After consumption, it can elevate blood glucose response levels, and further induce obesity and metabolic syndrome, limiting its application in the field of functional ingredient encapsulation. Additionally, since the internal pore size of natural maize starch is much larger than the hydrodynamic volume of polyphenol compounds, there is often a burst release when polyphenol compounds are released via maize starch encapsulation systems. Excessively rapid and premature release will lead to excessive accumulation of polyphenol compounds in a short period of time, which will lead to a series of side reactions and affect its ultimate efficacy. Based on the structural characteristics of natural maize starch, the maize starch-polyphenol complex prepared by the prior art suffers from the drawback of burst release.

Therefore, there is an urgent need to provide a method for preparing a sustained-release polyphenol maize starch.

### SUMMARY OF THE INVENTION

The purpose of the present invention is to provide an intestinal-targeted sustained-release polyphenol/resistant starch and a preparation method and application thereof, which solves the problem of burst release when polyphenol compounds are released through a maize starch encapsulation system in the prior art.

The technical solution adopted by the present invention is: the present invention provides a method for preparing intestinal-targeted sustained-release polyphenol/resistant starch, comprising the following steps:

Prepare quinoa polyphenol extract; anneal and recrystallize heat-dispersed high-amylose maize starch in an ethanol solution to obtain single-helix resistant maize starch; self-assemble the single-helix resistant maize starch and quinoa polyphenol extract in an alcohol-water system to obtain intestinal-targeted sustained-release polyphenol/resistant starch.

Preferably, the quinoa polyphenol extract comprises one or more of the following: white quinoa polyphenol extract, red quinoa polyphenol extract and black quinoa polyphenol extract. The extract is obtained by extracting white quinoa seeds, red quinoa seeds or black quinoa seeds using a methanol solution containing formic acid.

Preferably, the content of amylose in the high-amylose maize starch is 65%.

Preferably, the process of annealing and recrystallizing the heat-dispersed high-amylose maize starch in an ethanol solution comprises as follows: dissolving the high-amylose maize starch in an aqueous DMSO solution in a boiling water bath to obtain a first heat-dispersed system, washing the cooled first heat-dispersed system (at room temperature) with anhydrous ethanol, collecting a first precipitate, drying the first precipitate, crushing and sieving, dissolving the crushed and sieved first precipitate in an aqueous ethanol solution to obtain a second dispersion system, washing the second dispersion system, collecting a second precipitate, drying the second precipitate, and crushing and sieving.

Preferably, the process of self-assembly of the single helical structure resistant maize starch and quinoa polyphenol extract in the alcohol-water system is: mixing the single helical structure resistant maize starch with the ethanol-water solution, adding the quinoa polyphenol extract, mixing well to obtain a third dispersed system, washing the cooled third dispersed system (at room temperature), collecting the third precipitate, drying the third precipitate, and crushing and sieving.

Preferably, the ethanol aqueous solution is 40% aqueous ethanol by volume.

The present invention also provides an intestinal-targeted sustained-release polyphenol/resistant starch prepared by the preparation method.

Preferably, the intestinal-targeted sustained-release polyphenol/resistant starch is used to prepare food or medicine for preventing and/or treating obesity.

Preferably, the intestinal-targeted sustained-release polyphenol/resistant starch is used for preparing food or pharmaceuticals for lowering blood glucose levels.

Preferably, the intestinal-targeted sustained-release polyphenol/resistant starch is used to prepare food or medicine for protecting the intestinal barrier. Preferably, the intestinal-targeted sustained-release polyphenol/resistant starch is used in the preparation of food or pharmaceuticals for modulating intestinal microbiota.

Compared with the prior art, the present invention has the following beneficial effects:

The present invention provides a method for preparing intestinal-targeted sustained-release polyphenol/resistant starch, the steps comprising:

Prepare quinoa polyphenol extract; anneal and recrystallize heat-dispersed high-amylose maize starch in an ethanol solution to obtain single-helix resistant maize starch; self-assemble the single-helix resistant maize starch and quinoa polyphenol extract in an alcohol-water system to obtain intestinal-targeted sustained-release polyphenol/resistant starch.

The intestinal-targeted sustained-release polyphenol/resistant starch prepared by the present invention exhibits a specific starch helical structure, enabling intestinal-targeted controlled release of polyphenols. This structure reduces the degradation of phenolic compounds during gastrointestinal digestion, achieve accurate and sustained-release of polyphenol compounds during gastrointestinal digestion, and prolongs the retention time of polyphenols in the intestine, thereby improving its bioavailability. Additionally, the formed complex acts as a prebiotic to promote the increase in the abundance of beneficial microorganisms (e.g., Lactobacillus, Bifidobacterium and Akkermansia) in the colon, increases the production of short-chain fatty acids such as acetic acid and butyric acid, and reduces the relative abundance of harmful bacteria (e.g., Desulfovibrionaceae), demonstrating significant prebiotic properties.

The present invention forms a single-helix structure resistant maize starch-quinoa polyphenol complex system in an alcohol-water system, which is used to regulate the diameter of the starch single helical pores and to interact with different small molecule polyphenol compounds, thereby regulating the release rate of the polyphenol compounds. Specifically, the starch single helical structure can form multiple interactions (e.g., π-π interactions, hydrogen bonds, and electrostatic interactions) with the polyphenol compounds, thereby achieving effective loading of the polyphenol compounds. This complex system is cost-effective and free from toxic side effects.

The multi-scale single-helix pore structure formed in the self-assembly system of the single-helix resistant maize starch-quinoa polyphenol complex significantly increases the ordered crystalline content of starch, thereby delaying its hydrolysis by digestive enzymes. The encapsulation of polyphenol compounds within the single-helix structure effectively prevents their degradation during gastrointestinal digestion, enhancing bioavailability.

The present invention is based on the single helical structure of resistant maize starch-quinoa polyphenol complex can be partially or completely degraded by the intestinal microbiota in the colon, thereby triggering the release of polyphenol compounds in the target site. Additionally, resistant starch acts as a prebiotic, providing additional health benefits such as modulating the composition of gut microbiota and increasing short-chain fatty acids production, thereby enhancing practical utility.

The single-helix structure resistant maize starch-quinoa polyphenol complex sustained-release system prepared in this invention entirely avoids burst release during gastric digestion, with an average daily cumulative polyphenol release of less than 10%.

The preparation method of the present invention is simple and cost-effective for industrial-scale production. The materials used are natural bio-based materials with high safety. The obtained single-helix structure resistant maize starch-quinoa polyphenol complex can maintain structural stability during gastrointestinal digestion and is suitable for the field of polyphenol compound delivery.

Other advantages, objectives and features of the present invention will be embodied in part through the following description, and in part will be understood by those skilled in the art through study and practice of the present invention.

### BRIEF DESCRIPTION OF THE DRAWINGS

FIG. 1 is a ¹³C CP/MAS NMR spectrum of the native maize starch prepared in Comparative Example 1 and the single-helix resistant maize starch prepared in S2 in Example 1. FIG.

FIG. 2 shows the encapsulation efficiency of the maize starch-quinoa polyphenol complex prepared in Comparative Example 5 and the single-helix structure resistant maize starch-quinoa polyphenol complex prepared in Example 4.

FIG. 3 is an X-ray diffraction diagram, A is the X-ray diffraction diagram of the natural maize starch prepared in Comparative Example 1 and the single-helix structure resistant maize starch prepared by S2 in Example 1; B is the X-ray diffraction diagram of the single-helix structure resistant maize starch-quinoa complex prepared in Examples 1 to 3.

FIG. 4 shows the final accumulated quinoa polyphenol release efficiency of the maize starch-quinoa polyphenol complex prepared in Comparative Example 5 and the single-helix structure resistant maize starch-quinoa polyphenol complex prepared in Example 4.

FIG. 5 shows the concentration of polyphenols in serum at different times after intragastric administration of the maize starch-quinoa polyphenol complex prepared in Comparative Example 5 and the single-helix resistant maize starch-quinoa polyphenol complex prepared in Example 4.

FIG. 6 shows the changes in mice in each treatment group, A: changes in fasting blood glucose; B: changes in body weight.

FIG. 7 shows the 16S rRNA sequencing results of mouse fecal flora, A and B: the relative abundance of intestinal microbiota of each group of mice at the phylum level and genus level, respectively.

FIG. 8 shows the content of short-chain fatty acids SCFAs in the colon contents of mice, A˜F: the content of acetic acid, propionic acid, butyric acid, isobutyric acid, valeric acid and isovaleric acid, respectively.

### DETAILED DESCRIPTION

The present invention is further described below by specific examples, but the scope of the present invention is not limited thereto. The details and forms of the technical solution of the present invention may be modified or replaced without departing from the spirit and scope of the present invention, but these modifications or replacements all fall within the protection scope of the present invention.

The inventive concept of the present invention is as follows:

Existing technology shows that natural maize starch, as the external matrix for encapsulating polyphenols, is highly digestible and easily hydrolyzed by digestive enzymes. In addition, its internal pore size is much larger than the hydrodynamic volume of polyphenol compounds. Therefore, when polyphenol compounds are released through the natural maize starch encapsulation system, an explosive release often occurs. Such excessively rapid and premature release may lead to excessive accumulation of polyphenol compounds within a short period, resulting in a series of adverse reactions.

Based on this, the present invention proposes a method for preparing intestinal-targeted sustained-release polyphenol/resistant starch, the steps comprising: preparing quinoa polyphenol extract; annealing and recrystallizing heat-dispersed high-amylose maize starch in an ethanol solution to obtain single-helix structure resistant maize starch; self-assembling the single-helix structure resistant maize starch and quinoa polyphenol extract in an alcohol-water system to obtain intestinal-targeted sustained-release polyphenol/resistant starch.

The present invention is based on the non-covalent interactions between polyphenol compounds and the single helical structure of maize starch during the self-assembly process. By adjusting the multi-scale structure of maize starch, a specific starch helical structure can be used to control the release of polyphenols within the inclusion complexes, thereby reducing the loss of active substances in gastrointestinal digestion. Simultaneously, polyphenol compounds promote dense stacking of the single helical starch structure, inducing starch to form an ordered crystalline architecture that resists hydrolysis by digestive enzymes. This process generates enzyme-resistant starch, which remains indigestible in the upper intestine but can be partially or completely degraded by colonic microorganisms, exhibiting prebiotic effects and enabling targeted release of polyphenols. A synergistic relationship may exist between polyphenol compounds and starch, collectively contributing to beneficial regulatory effects on the host. Therefore, the sustained and stable release of polyphenol compounds in the intestinal tract by constructing a new type of universal resistant starch single helical delivery system has considerable application prospects.

In order to enable those skilled in the art to better understand and implement the technical solution of the present invention, the present invention is further described below in conjunction with specific embodiments and drawings.

In the description of the present invention, unless otherwise specified, all reagents used are commercially available and all methods used are conventional techniques in the art.

### EXAMPLE 1

A method for preparing intestinal-targeted sustained-release polyphenol/resistant starch, comprising the following steps:

S1: Prepare quinoa polyphenol extract as follows:

60-mesh powder, the seeds of quinoa were ultrasonically extracted with 80% (v/v) methanol at room temperature for 40 min. The methanol also contained 0.1% (v/v) formic acid. The mass volume ratio of quinoa seeds to 80% methanol was 1:25 (w/v). The extraction process was repeated twice, and the supernatants were combined and rotary evaporated at 45°° C. The concentrated solution was freeze-dried, i.e., the quinoa polyphenol extract, and stored at −20° C.

S2: The heat-dispersed high-amylose maize starch is annealed and recrystallized in an ethanol solution to obtain a single-helix resistant maize starch, as follows:

High amylose maize starch with an amylose content of 65% was dissolved in a 95% aqueous DMSO solution under boiling water bath conditions, and stirred continuously in the boiling water bath for 1.5 hours to obtain a thermal dispersion system, and the mass volume ratio of high amylose maize starch to DMSO aqueous solution was 1:25 (m/v). After cooling to room temperature, the thermal dispersion system was evenly mixed with 2.5 times the volume of anhydrous ethanol, and then the mixture was centrifuged at 5000 g for 10 min, the supernatant was discarded, the precipitate was washed twice with anhydrous ethanol, dried overnight at 37° C. in a vacuum drying oven at 5 Pa, and crushed through a 200-mesh sieve. Subsequently, the starch powder was bathed in a 40% (v/v) aqueous ethanol solution at 70° C. for 0.5 h, washed and dried with anhydrous ethanol, and ground to 200-mesh to obtain a single helixresistant maize starch, recorded as HAMS, and stored in a drying dish.

S3: The single helical resistant maize starch and quinoa polyphenol extract are self-assembled in an alcohol-water system to obtain a single helix resistant maize starch-quinoa polyphenol complex, as follows:

Prepared by S2 was mixed with a 40% (v/v) ethanol solution at a mass volume ratio of 1:100 (m/v), and the quinoa polyphenol extract prepared by S1 was added, and the amount of quinoa polyphenol extract added was 10% of the weight of the single helix resistant maize starch. Stirring was continued for 0.5 h in a 70° C. water bath, and after cooling to room temperature, it was mixed with 2.5 times the volume of anhydrous ethanol, and then the mixture was centrifuged at 5000 g for 10 min, the supernatant was discarded, and the precipitate was washed twice with anhydrous ethanol, dried overnight at 37° C. in a vacuum drying oven, and crushed through a 200-mesh sieve to obtain a single helixresistant maize starch-quinoa polyphenol complex, which was recorded as HAMS-WQP, i.e., intestinal-targeted sustained-release polyphenol/resistant starch, and stored in a drying dish for subsequent experimental determination.

### EXAMPLE 2

A method for preparing intestinal-targeted sustained-release polyphenol/resistant starch, comprising the following steps:

Red quinoa seeds were used to prepare red quinoa polyphenol extract. When preparing the single helix resistant maize starch-quinoa polyphenol complex, red quinoa polyphenol extract was used. The other processes were exactly the same as in Example 1. The obtained single helix resistant maize starch-quinoa polyphenol complex was recorded as HAMS-RQP, i.e., intestinal-targeted sustained-release polyphenol/resistant starch.

### EXAMPLE 3

A method for preparing intestinal-targeted sustained-release polyphenol/resistant starch, comprising the following steps:

Black quinoa seeds were used to prepare black quinoa polyphenol extract. When preparing the single helix resistant maize starch-quinoa polyphenol complex, the black quinoa polyphenol extract was used. The other processes were exactly the same as in Example 1. The obtained single helix resistant maize starch-quinoa polyphenol complex was recorded as HAMS-BQP, i.e., intestinal-targeted sustained-release polyphenol/resistant starch.

### EXAMPLE 4

A method for preparing intestinal-targeted sustained-release polyphenol/resistant starch, comprising the following steps:

According to S1 of Examples 1 to 3, white quinoa polyphenol extract, red quinoa polyphenol extract and black quinoa polyphenol extract were prepared. The white quinoa polyphenol extract, the red quinoa polyphenol extract and the black quinoa polyphenol extract were mixed in a weight ratio of 1:1:1 to prepare a mixed quinoa polyphenol extract, and the obtained single helix resistant maize starch-quinoa polyphenol complex was recorded as HAMS-QP. When preparing the single helical resistant maize starch-quinoa polyphenol complex, the mixed quinoa polyphenol extract was used, and the other processes were exactly the same as in Example 1.

In order to further illustrate the characteristics of the intestinal-targeted sustained-release polyphenol/resistant starch prepared by the method of the present invention, the present invention sets Comparative Examples 1 to 5.

### COMPARATIVE EXAMPLE 1

A method for preparing maize starch, comprising the following steps:

The natural maize starch was used as a control sample. The natural maize starch was dried in a vacuum drying oven overnight, ground to 200-mesh, recorded as NHAMS, and stored in a drying dish.

### COMPARATIVE EXAMPLE 2

A method for preparing a maize starch-quinoa polyphenol complex comprises the following steps:

S1: Prepare quinoa polyphenol extract using the same method as in Example 1.

S2: Prepare maize starch-quinoa polyphenol complex as follows:

The natural maize starch was mixed with a 40% (v/v) ethanol solution at a mass volume ratio of 1:100 (m/v), and the quinoa polyphenol extract prepared by S1 was added, and the amount of quinoa polyphenol extract added was 10% of the weight of the natural maize starch. Stirring was continued for 0.5 h in a 70° C. water bath, and after cooling to room temperature, it was mixed with 2.5 times the volume of anhydrous ethanol, and then the mixture was centrifuged at 5000 g for 10 min, the supernatant was discarded, and the precipitate was washed twice with anhydrous ethanol, dried overnight at 37° C. in a vacuum drying oven, and crushed through a 200-mesh sieve to obtain a maize starch-quinoa polyphenol complex, recorded as NHAMS-WQP, and stored in a drying dish for subsequent experimental determination.

### COMPARATIVE EXAMPLE 3

A method for preparing a maize starch-quinoa polyphenol complex comprises the following steps:

S1: Prepare red quinoa polyphenol extract using the same method as in Example 2.

S2: Prepare maize starch-quinoa polyphenol complex as follows:

The quinoa polyphenol added was red quinoa polyphenol extract, and the rest of the process was exactly the same as that of Comparative Example 2, to obtain a maize starch-quinoa polyphenol complex, which was recorded as NHAMS-RQP.

### COMPARATIVE EXAMPLE 4

A method for preparing a maize starch-quinoa polyphenol complex comprises the following steps:

S1: Prepare black quinoa polyphenol extract using the same method as in Example 3.

S2: Prepare maize starch-quinoa polyphenol complex as follows:

The quinoa polyphenol added was black quinoa polyphenol extract, and the rest of the process was exactly the same as that of Comparative Example 2, to obtain a maize starch-quinoa polyphenol complex, which was recorded as NHAMS-BQP.

### COMPARATIVE EXAMPLE 5

A method for preparing a maize starch-quinoa polyphenol complex comprises the following steps:

S1: preparing a mixed quinoa polyphenol extract of white quinoa polyphenol extract, red quinoa polyphenol extract and black quinoa polyphenol extract, the method is exactly the same as that of Example 4.

S2: Prepare maize starch-quinoa polyphenol complex as follows:

The quinoa polyphenol added was a mixed quinoa polyphenol extract, and the rest of the process was exactly the same as that of Comparative Example 2, to obtain a maize starch-quinoa polyphenol complex, which was recorded as NHAMS-QP

The single helix structure resistant maize starch-quinoa polyphenol complex prepared in Examples 1 to 4, the maize starch prepared in Comparative Example 1, and the maize starch-quinoa polyphenol complex prepared in Comparative Examples 2 to 5 were subjected to starch multi-scale structure test.

### (1) Helical Structure Test of Native Maize Starch and Single Helical Resistant Maize Starch.

The natural maize starch prepared in Comparative Example 1 and the single helix resistant maize starch prepared in S2 in Example 1 were collected using a solid-state nuclear magnetic spectrometer equipped with a 4-mm MAS probe. The operating conditions were set as follows: resonance frequency 188.6 MHz, rotor speed 10 kHz, contact time 1 ms, delay time 5 s, cumulative scanning times more than 2400 times, and all experiments were carried out at 25° C.

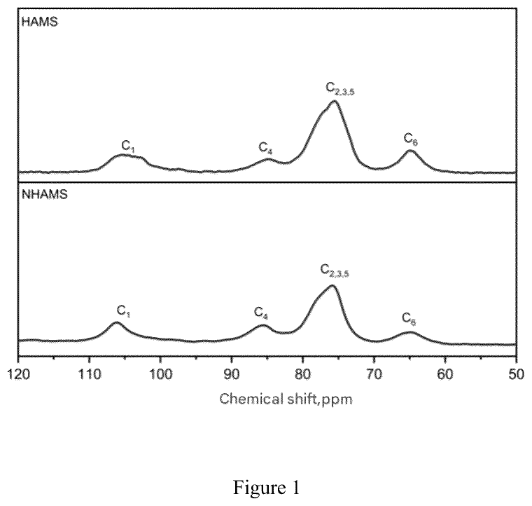

The results are shown in FIG. 1. Compared with the natural maize starch sample, the single-helix structure resistant maize starch HAMS sample showed a tiny single peak at 94 ppm˜105 ppm, which is characterized by a typical empty helical polymorph. This may be caused by the formation of a left-handed single helical structure, proving the successful preparation of single-helix structure resistant maize starch.

### (2) Encapsulation Efficiency Test of Maize Starch-Quinoa Polyphenol Complex and Single Helical Structure Resistant Maize Starch-Quinoa Polyphenol Complex.

The single helical resistant maize starch-quinoa polyphenol complex prepared in Example 4 and the maize starch-quinoa polyphenol complex prepared in Comparative Example 5 were subjected to encapsulation efficiency determination. 200 mg of the sample was mixed with 10 mL of ethanol to remove free polyphenol compounds, centrifuged at 5000 g for 10 min. The supernatant was collected, and the polyphenol content was quantified using the Folin-Ciocalteu method. The encapsulation efficiency (EE) was then calculated using the following equation:

$$Encapsulation efficiency ( \% ) = \frac{total phenolic content - supernatant phenolic content}{total phenolic content} \times 100 .$$

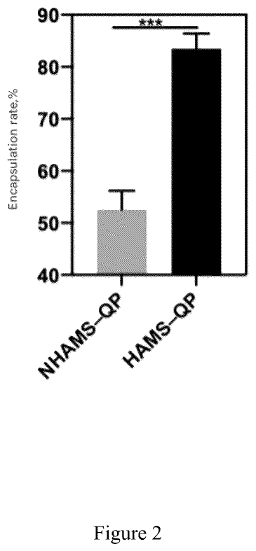

The results are shown in FIG. 2. Compared with the maize starch-quinoa polyphenol complex, the single-helix resistant maize starch-quinoa polyphenol complex showed a higher encapsulation efficiency, indicating the efficient loading and encapsulation of polyphenol compounds by the single-helix resistant maize starch.

### (3) Crystal Structure Test of Native Maize Starch, Single Helix Resistant Maize Starch and Single Helix Resistant Maize Starch-Quinoa Polyphenol Complex.

The single helix resistant maize starch-quinoa polyphenol composite prepared in Examples 1 to 3, the natural maize starch prepared in Comparative Example 1, and the single helical resistant maize starch prepared in S2 in Example 1 were subjected to X-ray diffraction measurement using an X-ray diffractometer at 40 kV and 40 mA, with Cu-Ka radiation as the X-ray source. The diffraction angle 20 ranged from 5° C. to 50° C., and the scanning speed was 10° C./min.

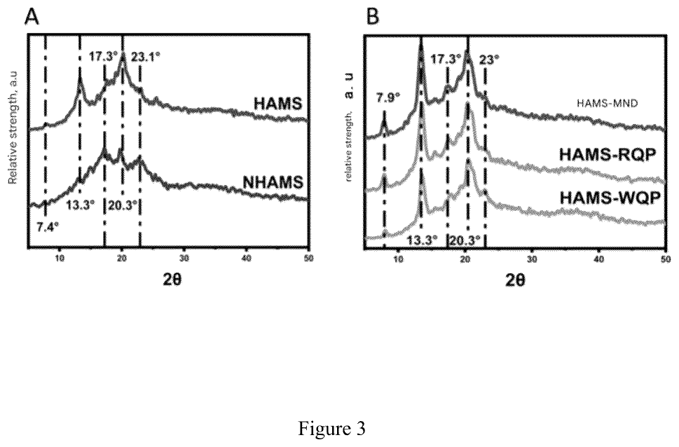

The results are shown in FIG. 3. Compared with natural maize starch, single-helix resistant maize starch exhibits typical V-type crystallization peaks at 7.4° C., 13.3° C. and 20.3° C., and has the potential to accommodate guest molecular phenolic compounds. Compared with single-helix resistant maize starch, single-helix resistant maize starch-quinoa polyphenol complex forms sharper diffraction peaks, indicating that a more ordered structure has been formed.

### (4) Testing the Release Characteristics of Phenolic Compounds in Single-Helix Resistant Maize Starch-Quinoa Polyphenol Complex During in Vitro Simulated Gastrointestinal Digestion.

The release profiles of the single-helix resistant maize starch-quinoa polyphenol complex (prepared in Example 4) and the maize starch-quinoa polyphenol complex (prepared in Comparative Example 5) were tested. During the simulated gastric digestion phase, digestive fluid samples were collected at 5, 10, 20, 30, 60, 90, and 120 minutes, while in the simulated intestinal digestion phase, samples were collected at 5, 10, 20, 30, 60, 90, 120, 240, 360, 480, and 600 minutes. After collection, the enzymes were inactivated, and the samples were centrifuged at 5000 g for 10 min. The supernatant was collected, and polyphenol content was quantified using the Folin-Ciocalteu method.

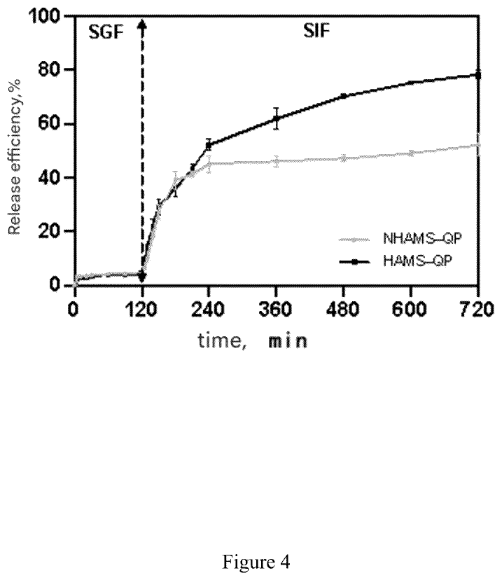

The results are presented in FIG. 4. During the gastric digestion stage, 4.5% of phenolic compounds were released from the maize starch-quinoa polyphenol complex, compared to 4.3% from the single-helix resistant maize starch-quinoa polyphenol complex. Upon entering the intestinal phase, rapid release of quinoa polyphenols occurred from the maize starch complex, attributed to enzymatic hydrolysis of starch by digestive enzymes. In contrast, the single-helical resistant complex exhibited sustained polyphenol release even after 2 h of intestinal digestion. The ordered helical structure of HAMS delayed degradation kinetics and protected cavity-encapsulated polyphenols from enzymatic and chemical degradation in digestive fluids. These findings demonstrate the pH-responsive intestinal release behavior and controlled-release properties of HAMS.

(5) The single-helix resistant maize starch-quinoa polyphenol complex prepared in Example 4 and the maize starch-quinoa polyphenol complex prepared in Comparative Example 5 were subjected to a pharmacokinetic test of phenolic substances in rats.

Six-week-old male Sprague-Dawley (SD) rats were obtained from Hangzhou Medical College. The animals were housed under a 12-h light/dark cycle at 25° C. and 60% relative humidity, with ad libitum access to standard rodent chow and water. All experimental procedures were approved by the Animal Ethics Committee of Zhejiang Province and conducted in compliance with the Guide for the Care and Use of Laboratory Animals.

After a 1-week acclimatization period, rats were randomly allocated into three groups: 1) blank control group, 2) model group, and 3) experimental group. All animals were fasted for 12 h prior to pharmacokinetic testing. Body weight-adjusted oral gavage was administered at a dose of 30 mg/kg to all groups: Blank control group: Received 1 mM phosphate-buffered saline (PBS, pH 7.4); Model group: Received the maize starch-quinoa polyphenol complex (Comparative Example 4) dissolved in PBS; Experimental group: Received the single-helical resistant maize starch-quinoa polyphenol complex (Example 3) dissolved in PBS. Serum samples were collected via orbital sinus puncture at specified time points post-administration (5, 15, 30 min; 1, 2, 4, 8, 12, and 20 h). Samples were centrifuged at 3,000 g for 10 min to isolate serum. For phenolic extraction, 200 μL of acetonitrile was added to 100 μL of serum, followed by centrifugation at 13,000 g for 20 min. The supernatant was filtered through a 0.22-μm membrane and analyzed by ultra-high-performance liquid chromatography (UPLC).

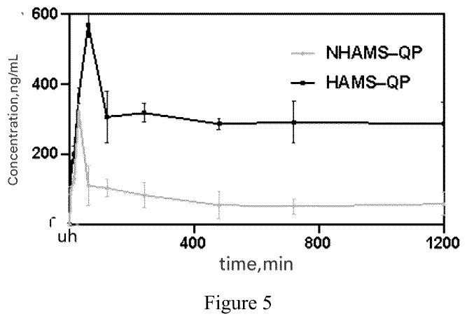

The results are shown in FIG. 5. Rats were administered PBS solutions containing equivalent phenolic content from the maize starch-quinoa polyphenol complex and the single-helical resistant maize starch-quinoa polyphenol complex. Serum concentrations of phenolic compounds were quantified at different time points and the pharmacokinetic parameters were calculated. The maximum blood drug concentration Cₘₐₓ of quinoa polyphenol in the single-helix resistant maize starch-quinoa polyphenol complex group appeared at 60 min after gavage, while the maximum blood drug concentration Cₘₐₓ of maize starch-quinoa polyphenol complex appeared at 15 min. The single-helical resistant starch matrix enabled sustained release of phenolic compounds, maintaining a stable serum concentration for a long time. These results are consistent with the results of phenolic substance release in simulated gastrointestinal digestion, confirming the regulatory ability of single helical resistant maize starch-quinoa polyphenol complex to protect the release of phenolic compounds.

(6) In Vivo Experiments Were Conducted on Mice Using the Single-Helix Resistant Maize Starch-Quinoa Polyphenol Complex Prepared in Examples 1 To 3 and The Single-Helix Resistant Maize Starch Prepared in S2 In Example 1.

### 6.1 The Feeding and Grouping of Mice in Animal Experiments are as Follows:

Six-week-old male C57BL/6J mice were purchased from Hangzhou Medical College. The feeding conditions were 12 h light cycle, 25° C., 60% relative humidity, and the animals had free access to mouse feed and drinking water. After 1 week of adaptation, 36 mice were randomly divided into 6 groups, 6 mice/group, including a normal control group, a model group, and 4 experimental groups. The normal control group CD was given a basal diet; the model group HFD was given a high-fat diet; the four dietary intervention experimental groups of HFD^{HAMS}, HFD^{HAMS-WQP}, HFD^{HAMS-RQP} and HF_{DHAMS-BQP} were fed with 20% of the single-helix resistant maize starch prepared in S2 of Example 1, and the single-helix resistant maize starch-quinoa polyphenol complex prepared in Examples 1 to 3. The carbohydrate replacement amount of HFD, that is, 20% of the mass of carbohydrates in the mouse feed ingredients was replaced by HAMS or HAMS-QP. Therefore, the maize starch mass in the carbohydrates of the model group was 72.8 gm; the maize starch mass in the carbohydrates of the dietary intervention group was 58.3 gm %, plus 14.5 gm % of single-helix resistant maize starch or single-helix resistant maize starch-polyphenol complex. All groups were fed for 8 weeks, and the specific feed formula is shown in Table 1.

TABLE 1
Mouse feed composition

| | | | Dietary |
|---|---|---|---|
| | | | intervention |
| | Normal | Model | group - 20% |
| | control | group | carbohydrate |
| | group CD - | HFD- | replacement |
| | conventional | high | of high- |
| | feed, | fat diet, | fat diet, |
| Element | gm % | gm % | gm % |
| Casein | 200 | 200 | 200 |
| L-Cystine | 3 | 3 | 3 |
| Maize starch | 506.2 | 72.8 | 58.3 |
| HAMS/ | — | — | 14.5 |
| HAMS-QPs | | | |
| Maltodextrin | 125 | 100 | 100 |
| sucrose | 72.8 | 176.8 | 176.8 |
| Cellulose | 50 | 50 | 50 |
| Soybean Oil | 25 | 25 | 25 |
| lard | 20 | 177.5 | 177.5 |
| Mixed Minerals | 50 | 50 | 50 |
| S10026B | | | |
| Mixed Vitamins | 1 | 1 | 1 |
| V10001C | | | |
| Choline bitartrate | 2 | 2 | 2 |

Note:
“—” means there is no such item.

### 6.2 Mouse Body Weight and Fasting Blood Glucose Were Measured as Follows:

The weight and fasting blood glucose levels of mice were measured every week. After fasting for 12 hours, the fasting blood glucose levels of the tail vein blood of mice were measured by a blood glucose meter.

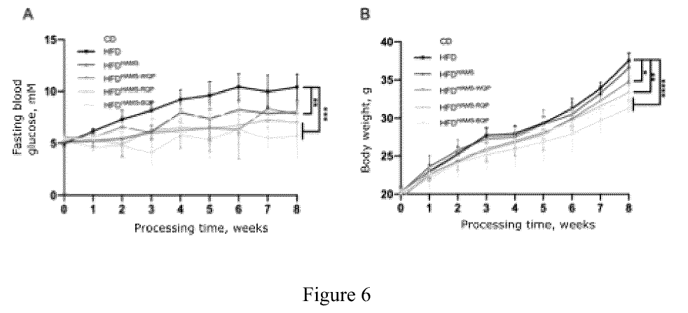

As illustrated in FIG. 6, after an 8-week dietary intervention, the high-fat diet (HFD) group exhibited significantly greater weight gain compared to the control diet (CD) group, with a >10% increase in body weight (p<0.05), confirming successful establishment of the HFD-induced obesity model. Notably, the HFD^{HAMS-BQP} intervention group showed a markedly lower mean body weight than both the HFD group and the CD group (p<0.01). To evaluate the antihyperglycemic effects of the single-helix resistant maize starch-quinoa polyphenol complex (HAMS-QP), fasting blood glucose (FBG) levels were measured. The HFD group displayed significantly elevated FBG levels relative to the CD group (p<0.001). In contrast, dietary interventions with HFD^{HAMS}, HFD^{HAMS-WQP}, HFD^{HAMS-RQP}, and HFD^{HAMS-BQP} significantly attenuated hyperglycemia (p<0.05 vs. HFD), with HFD^{HAMS-RQP} and HFDHAMS-BQP demonstrating superior efficacy (p<0.01). These findings collectively indicate that the single-helix resistant maize starch (HAMS) and its polyphenol complex (HAMS-QP) exert anti-obesity effects in HFD-induced obese mice while concurrently reducing hyperglycemia, suggesting dual metabolic benefits.

6.3 16S rRNA Sequencing of Mouse Fecal Flora is as Follows:

For gut microbiota analysis, total DNA was extracted from mouse fecal samples. Following assessment of DNA concentration and integrity, the V3-V4 region of the 16S rRNA gene was PCR-amplified and sequenced on the Illumina MiSeq platform. Raw sequences were processed through quality filtering, alignment, and clustering using MOTHUR v.1.45.3. Chimeric sequences were identified and removed using UPARSE v.11 and UCHIME v.4.2. High-quality reads were clustered into operational taxonomic units (OTUs) at a 97% similarity threshold. Taxonomic classification of OTUs was performed with the Ribosomal Database Project (RDP) Classifier v.2.13 (minimum confidence: 0.6), integrated within the QIIME v1.9.1 pipeline. Linear discriminant analysis effect size (LEfSe) was employed to identify statistically significant and biologically discriminative taxa. Statistical significance of species or functional differences was determined using R v3.4.1, with the Wilcoxon rank-sum test.

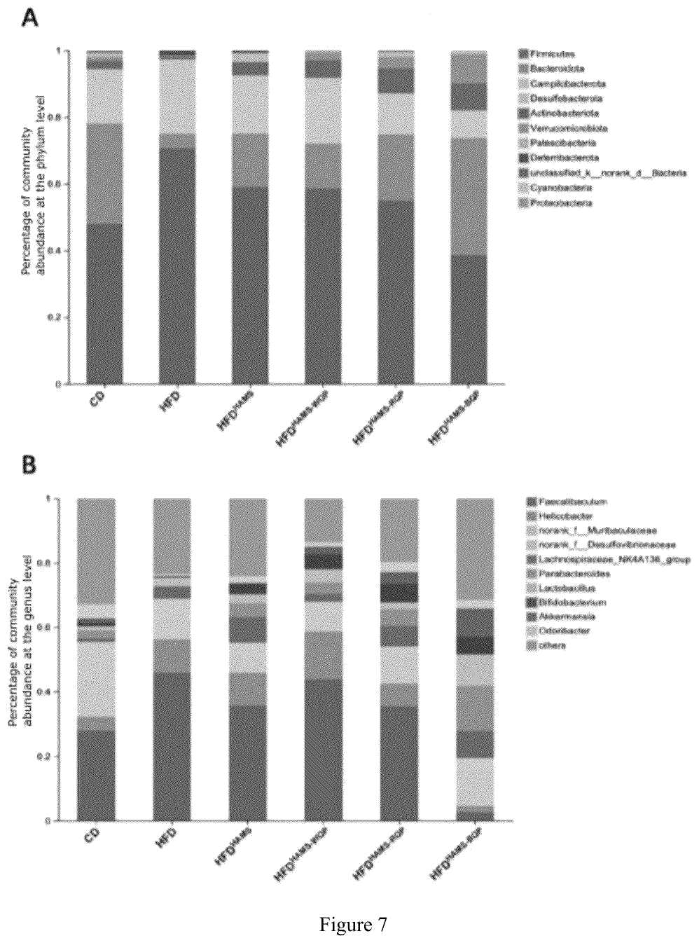

In order to evaluate the effects of single helical resistant maize starch and single-helix resistant maize starch-quinoa polyphenol complex on the intestinal microbial composition of HFD-induced obese mice, the relative abundance of taxa in different groups was compared. As shown in FIG. 7, at the phylum level, the relative abundance of Firmicutes and Bacteroidetes changed significantly. The ratio of Firmicutes to Bacteroidetes is considered to be highly correlated with the risk of obesity, and studies have reported that an increase in the F/B value is also one of the inducing factors of inflammatory bowel disease. The F/B value of HFD-induced obese mice increased significantly compared with the CD group, while the F/B value of mice in the single-helix resistant maize starch-quinoa polyphenol complex dietary intervention group was reduced to varying degrees. In order to further analyze the intervention and regulatory effects of the single-helix resistant maize starch-quinoa polyphenol complex on the intestinal microbiota of HFD-induced obese mice, the intestinal microbiota of mice was evaluated at the genus level. Akkermansia and other probiotics play an important role in protecting the intestinal barrier function. As shown in FIG. 7B, after the single-helix resistant maize starch-quinoa polyphenols complex dietary intervention, the relative abundance of microbiota including beneficial Lactobacillus, Bifidobacterium and Akkermansia increased; in addition, the relative abundance of short-chain fatty acid-producing bacteria including Bifidobacterium and Lachnospiraceae increased. Studies have shown that the intake of dietary fiber such as resistant starch can effectively reduce the relative abundance of Desulfovibrionaceae and other genera. Desulfovibrionaceae, as a sulfate-reducing bacteria, usually increases in ulcerative colitis hosts. The lipopolysaccharide produced by it can damage the intestinal barrier and further cause metabolic disorders and inflammatory responses in the body. The hydrogen sulfide product produced by reducing sulfate is a recognized epithelial cell toxic product, which will lead to abnormal proliferation and metabolism of intestinal epithelial cells and thus damage the intestinal barrier function. After dietary intervention with single-helix resistant maize starch-quinoa polyphenols complex, the abundance of the conditionally pathogenic bacteria genus Desulfovibrionacea was significantly reduced compared with the HFD group. The above facts indicate that dietary intervention with single-helix resistant maize starch-quinoa polyphenols complex significantly increased the relative abundance of beneficial bacteria, which to a certain extent reflects the probiotic characteristics of single-helix resistant maize starch-quinoa polyphenols complex.

### 6.4 Determination of the Content of Short-Chain Fatty Acids (SCFAs) in the Intestinal Contents of Mice, as Follows:

Freeze-dried mouse fecal samples were homogenized in phosphate-buffered saline (PBS) using a freeze-grinding protocol, followed by ultrasonication (10 min) and centrifugation at 13,000 g for 15 min at 4° C. The supernatant was mixed with n-butanol solvent (1:1 v/v), vortexed for 10 s, and ultrasonicated at 4° C. for 10min. After centrifugation under identical conditions, the organic phase was transferred to GC-MS vials for analysis. SCFA concentrations (acetic acid, propionic acid, butyrate acid, isobutyric acid, valeric acid, isovaleric acid) were calculated using external standard curves generated in Agilent MassHunter Quantitative Analysis software (v.B.10.0), with automated peak integration and manual verification.

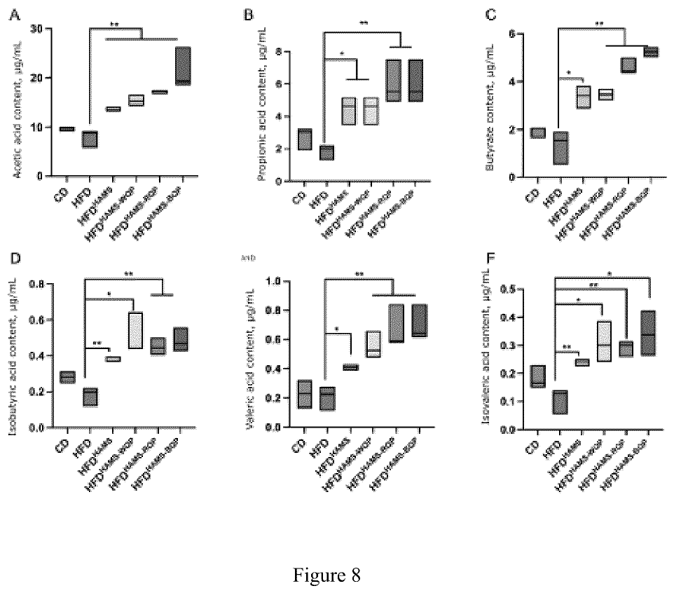

To further verify the probiotic properties of the single-helix resistant maize starch-quinoa polyphenol complex, SCFAs were identified and quantified in the intestinal contents of mice. As shown in FIG. 8, acetate accounts for the largest proportion of SCFAs, followed by propionate and butyrate. In particular, the HFD HAMS-B dietary intervention group significantly increased the SCFAs content in the intestinal contents of mice. After dietary intervention with single-helix resistant maize starch-quinoa polyphenol complex, the relative abundance of Bifidobacterium in the intestinal microbiota of mice increased significantly, which is potentially related to the production of SCFAs. It is inferred that the single-helix resistant maize starch-quinoa polyphenol complex increases the production of SCFAs by increasing the abundance of SCFAs-producing bacteria, and promotes the growth of host-friendly intestinal microbiota, further exerting its anti-inflammatory physiological function in the intestine and alleviating intestinal and systemic circulatory inflammation. These findings provide novel in vivo evidence validating that the quinoa polyphenol-starch complex functions as a novel resistant starch system, thereby addressing the knowledge gap in its in vivo characterization.

The technical features of the above-described embodiments may be arbitrarily combined. To make the description concise, not all possible combinations of the technical features in the above-described embodiments are described. However, as long as there is no contradiction in the combination of these technical features, they should be considered to be within the scope of this specification.

The above-mentioned embodiments only express several implementation methods of the present invention, and the description is relatively specific and detailed, but it cannot be understood as limiting the scope of the invention patent. It should be pointed out that for ordinary technicians in this field, several modifications and improvements can be made without departing from the concept of the present invention, which all belong to the protection scope of the present invention.

1. A method for preparing intestinal-targeted sustained-release polyphenol/resistant starch, characterized in that it comprises the following steps:
preparing a quinoa polyphenol extract;
annealing and recrystallizing heat-dispersed high-amylose maize starch in an ethanol solution to obtain single-helical resistant maize starch;
allowing single helical resistant maize starch and quinoa polyphenol extract to self-assemble in an alcohol-water system to obtain intestinal-targeted sustained-release polyphenol/resistant starch.

2. The preparation method according to claim 1, characterized in that the quinoa polyphenol extract is one or more of white quinoa polyphenol extract, red quinoa polyphenol extract and black quinoa polyphenol extract;
wherein the quinoa polyphenol extract is obtained by extracting white quinoa seeds, red quinoa seeds or black quinoa seeds using a methanol solution containing formic acid.

3. The preparation method according to claim 1, characterized in that the content of amylose in the high-amylose maize starch is 65%.

4. The preparation method according to claim 1, characterized in that the process of annealing and recrystallizing the heat-dispersed high-amylose maize starch in an ethanol solution is:
Dissolve high-amylose maize starch in an aqueous DMSO solution in a boiling water bath to obtain a first thermal dispersion system, wash the first thermal dispersion system cooled to room temperature with anhydrous ethanol, collect a first precipitate, dry the first precipitate, crush and sieve, dissolve the crushed and sieved first precipitate in an aqueous ethanol solution to obtain a second dispersion system, wash the second dispersion system, collect a second precipitate, dry the second precipitate, and crush and sieve.

5. The preparation method according to claim 1, characterized in that the process of self-assembly of the single helical resistant maize starch and quinoa polyphenol extract in an alcohol-water system is:
the single helical structure resistant maize starch is mixed with an aqueous ethanol solution, and quinoa polyphenol extract is added and mixed to obtain a third dispersion system. The third dispersion system cooled to room temperature is washed and a third precipitate is collected. The third precipitate is dried, crushed and sieved.

6. An intestinal-targeted sustained-release polyphenol/resistant starch prepared by the preparation method as claimed in claim 1.

7. The use of the intestinal-targeted sustained-release polyphenol/resistant starch according to claim 6, characterized in that the intestinal-targeted sustained-release polyphenol/resistant starch is used to prepare food or pharmaceutical composition for preventing and/or treating obesity.

8. The use of the intestinal-targeted sustained-release polyphenol/resistant starch according to claim 6, characterized in that the intestinal-targeted sustained-release polyphenol/resistant starch is used to prepare food or pharmaceutical composition for reducing blood glucose levels.

9. The use of the intestinal-targeted sustained-release polyphenol/resistant starch according to claim 6, characterized in that the intestinal-targeted sustained-release polyphenol/resistant starch is used to prepare food or pharmaceutical composition for protecting the intestinal barrier.

10. The use of the intestinal-targeted sustained-release polyphenol/resistant starch according to claim 6, characterized in that the intestinal-targeted sustained-release polyphenol/resistant starch is used to prepare food or pharmaceutical composition for modulating intestinal microbiota.
